(19) United States
(12) Patent Application Publication
(10) Pub. No.: US 2023/0236170 A1
(43) Pub. Date: Jul. 27, 2023

(54) REACTIVE AFFINITY PROBE-INTERACTION DISCOVERY PLATFORM

(71) Applicant: Jnana Therapeutics Inc., Boston, MA (US)

(72) Inventors: Matthew T. Labenski, Chelmsford, MA (US); Terry J. Rettenmaier, Edwardsville, IL (US); Lyn H. Jones, Winchester, MA (US); Giovanni Muncipinto, Cambridge, MA (US)

(21) Appl. No.: 17/769,173

(22) PCT Filed: Oct. 15, 2020
(86) PCT No.: PCT/US20/55657

§ 371 Date: Apr. 14, 2022

Related U.S. Application Data
(60) Provisional application No. 62/915,310, filed on Oct. 15, 2019.

(51) Int. Cl. G01N 33/50; G01N 33/68

(52) U.S. Cl. CPC G01N 33/5038; G01N 33/6845; G01N 2500/10

(57) ABSTRACT

Disclosed are methods, assays, and kits for identifying a ligand to a biological molecule, such as a protein, a lipid, a carbohydrate, or a nucleic acid. The disclosed methods may be a quantitative binding assay against targets, which sidesteps the challenge of target purification, and may provide a systematic approach to discover and target allosteric binding sites.

### RELATED APPLICATIONS

This application claims the benefit of priority to U.S. Provisional Patent Application Ser. No. 62/915,310, filed Oct. 15, 2019.

### BACKGROUND

Identifying “druggable” targets and their corresponding therapeutic agents are two fundamental challenges in drug discovery research. The pharmacology of many biological molecules, such as proteins, remains inaccessible as their endogenous or exogenous modulators have not been discovered. Tools that explore the physiological functions and pharmacological potential of these biological molecules, whether they are endogenous and/or surrogate ligands, are therefore of paramount importance.

The discovery of pharmacological modulators for a biological macromolecule of interest is often achieved by screening libraries of molecules for their capacity to bind to or functionally modulate the purified macromolecule. However, many classes of biological macromolecules, including multi-pass transmembrane proteins, are recalcitrant to purification from cells and must be screened in their native cellular environment. Such cell-based screening requires the selection of a surrogate readout, such as cell viability, signal transduction, or gene expression. Compound screening using surrogate cell-based readouts creates two challenges: (i) many hits will exert their pharmacology indirectly through off-target effects and (ii) many true ligands that alter other aspects of the macromolecule's function will be missed. A technology that enables the direct identification of small-molecule binders to a biological macromolecule of interest in intact cells would address these outstanding challenges and represent a significant advance for the field.

### SUMMARY OF THE INVENTION

The present invention provides a method and system for screening of ligand candidates for biological molecules. The reactive affinity probe interaction discovery (RAPID) technology is a quantitative binding assay against targets in situ, which sidesteps the challenge of target purification and may provide a systematic approach to discover and target allosteric binding sites.

Disclosed herein, in certain embodiments, is a method of identifying a ligand, comprising: (a) contacting a biological molecule and a probe molecule; wherein the probe molecule comprises a binding element, a reporter group, and a reactive moiety; the probe molecule binds to the biological molecule via the binding element; and the reactive moiety forms a covalent bond with the biological molecule, thereby forming a conjugate; (b) contacting the conjugate and a detectable molecule comprising a functional moiety that reacts with the reporter group, thereby forming a detectable conjugate; (c) contacting the detectable conjugate and a solid support; wherein the solid support comprises a recognition moiety; and the recognition moiety binds to the detectable conjugate, thereby forming a bound detectable conjugate; and (d) detecting the bound detectable conjugate, thereby identifying the probe molecule as a ligand for the biological molecule.

Numerous embodiments are further provided that can be applied to any aspect of the present invention described herein. For example, in some embodiments, the binding element is a small molecule, a peptide, or a nucleic acid (such as RNA or DNA). In some embodiments, the binding element is a component of a library comprising a plurality of binding elements. In some embodiments, the library comprises a library of small-molecule fragments that can be defined as satisfying the Rule of 3: molecular weight ≤300 Da, c Log P ≤3, hydrogen bond donors ≤3, and hydrogen bond acceptors ≤3. Exemplary libraries include: ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, the biological molecule is a protein. In some embodiments, the reactive moiety forms a covalent bond with an amino acid of the protein. In some embodiments, the biological molecule is a lipid, a carbohydrate, or a nucleic acid (such as RNA or DNA). In some embodiments, the biological molecule comprises an epitope tag, for example, FLAG, 6×His, HA, c-myc, glutathione-S-transferase, Strep-tag, maltose-binding protein, chitin-binding protein, S-tag, V5 tag, or AviTag. In some embodiments, the reporter group comprises an azadibenzocyclooctyne, a thiol, an alkene, an alkyne, an azide, a tetrazine, a trans-cyclooctene, a (diphenylphosphino)aryl, (diphenylphosphino)alkyl, or an activated ester (e.g., a hydroxybenzotriazole (HOBt) ester). In some embodiments, the reactive moiety is a photocrosslinker group, a sulfonyl fluoride, a fluorosulfate, a Michael acceptor moiety, a leaving group moiety, or a moiety that forms a covalent bond with a nucleophilic moiety in the side chain of a naturally occurring alpha amino acid (e.g., with the thiol group of a cysteine, the amino group of a lysine, the hydroxyl group of a serine or threonine, or the phenol group of a tyrosine). In some embodiments, the detectable molecule comprises digoxigenin, nickel NTA (nitrilotriacetic acid), a chromophore, or a luminophore. In some embodiments, the chromophore comprises non-fluorochrome chromophore, quencher, an absorption chromophore, fluorophore, organic dye, inorganic dye, metal chelate, or a fluorescent enzyme substrate. In some embodiments, the detectable molecule is biotin. The biotin can be bound to a streptavidin conjugate, such as, a HRP, a SulfoTag, a fluorophore, or a metal chelate. In some embodiments, the functional moiety comprises an azadibenzocyclooctyne, a thiol, an alkene, an alkyne, an azide, a tetrazine, a trans-cyclooctene, a (diphenylphosphino)aryl, (diphenylphosphino)alkyl, or an activated ester (e.g., a hydroxybenzotriazole (HOBt) ester). In some embodiments, the solid support is a membrane, glass, plastic, synthetically prepared polymer, an eppendorf tube, a well of a multi-well plate, or a surface plasmon resonance chip. In some embodiments, the recognition moiety is an antibody, a DNA binding protein, a RNA binding protein, a carbohydrate binding protein, or a lipid binding protein. In some embodiments, the antibody is an antibody against the biological molecule, and/or the epitope tag.

In some embodiments, step (d) comprises detecting the bound detectable conjugates via ELISA, Western blot, immunofluorescence assay, fluorometric assay, fluorometric microvolume assay technology (FMAT), or cell subcellular staining. In some embodiments, the method is performed on a crude cellular extract comprising the biological molecule, performed on a liposomal preparation of proteins comprising the biological molecule, performed on an isolated organelle comprising the biological molecule, performed on a purified protein preparation comprising the biological molecule, or performed in situ. In some embodiments, the method is a cell-based assay. In some embodiments, the biological molecule is expressed in a cell. In some embodiments, the cell is engineered to express the biological molecule. In some embodiments, the cell is lysed prior to step (b). In some embodiments, step (d) further comprises quantifying the amount of the bound detectable conjugate. In some embodiments, step (a) further comprises a substrate for the biological molecule. In some embodiments, the amount of the bound detectable conjugate formed in the presence of the substrate for the biological molecule is less than the amount of the bound detectable conjugate formed in the absence of the substrate (i.e., the probe molecule is a substrate-competitive probe). In other embodiments, the amount of the bound detectable conjugate formed in the presence of the substrate for the biological molecule is greater than the amount of the bound detectable conjugate formed in the absence of the substrate (i.e., the probe molecule is a substrate-cooperative probe).

### BRIEF DESCRIPTION OF THE DRAWINGS

FIG. 1 illustrates a general protocol for RAPID, a high-throughput screening, in cell binding assay for ligand discovery and target engagement.

FIGS. 2A-2C show that RAPID identifies orthosteric and allosteric ligands as starting points or probes for target occupancy. FIG. 2A shows that the extent of covalent modification of the creatine transporter by a subset of the RAP library is highly reproducible. FIG. 2B shows screen of 2000 RAPs against the creatine transporter SLC6A8±the substrate analog β-guanidinoproprionic acid (β-GPA). Dots that fall off-diagonal are either substrate-competitive or substrate-cooperative. FIG. 2C shows two RAPs identified from the screen show dose-dependent inhibition or enhancement of covalent modification of the target as a function of the concentration of GPA. The IC50/EC50 corresponds to β-GPA's known inhibition constant (˜30 μM).

FIG. 3 shows covalent inactivation of the creatine transporter SLC6A8 by the reactive affinity probe JN-1724 and protection by co-dosing with competitor β-GPA. Cells were dosed for 30 minutes with 100 μM JN-1724 with or without 1 mM β-GPA and then irradiated for 6 minutes with 365 nm light. Cells were washed and the residual transport activity of SLC6A8 was measured via a creatine uptake assay.

FIG. 4 shows mass spectrometry data that demonstrates covalent modification of SLC6A8 by the reactive affinity probe JN-1724 which is competed by co-dosing with β-GPA. Cells were dosed for 30 minutes with 20 μM JN-1724 with or without 1 mM β-GPA and then irradiated for 6 minutes with 365 nm light. Cells were lysed; biotin was clicked on; and biotinylated proteins were affinity-purified with streptavidin, digested with trypsin, and identified by tandem mass spectrometry with TMT quantification. SLC6A8 (dot pointed at with arrow) was one of the proteins identified and co-dosing with 1 mM β-GPA reduced the level of enrichment by 80%.

### DETAILED DESCRIPTION OF THE INVENTION

The present invention provides a method and system for screening of ligand candidates for biological molecules. The reactive affinity probe interaction discovery (RAPID) is a quantitative binding assay against targets in situ, which sidesteps the challenge of target purification and may provide a systematic approach to discover and target allosteric binding sites. The RAPID technology described herein enables the direct identification of small-molecule binders to a biological macromolecule of interest in intact cells.

Small molecules are powerful tools for investigating protein function and can serve as leads for new therapeutics. Most human proteins, however, lack small molecule ligands, and entire protein classes are considered undruggable. The method disclosed herein can identify small molecule probes for biological molecules, such as proteins, that have proven difficult to target using high-throughput screening of complex compound libraries. Although reversibly binding ligand are commonly pursued, covalent fragments provide an alternative route to small molecule probes, including those that can access regions of proteins that are difficult to target through binding affinity alone.

Disclosed herein, in certain embodiments, is a method of identifying a ligand, comprising: (a) contacting a biological molecule and a probe molecule; wherein the probe molecule comprises a binding element, a reporter group, and a reactive moiety; the probe molecule binds to the biological molecule via the binding element; and the reactive moiety forms a covalent bond with the biological molecule, thereby forming a conjugate; (b) contacting the conjugate and a detectable molecule comprising a functional moiety that reacts with the reporter group, thereby forming a detectable conjugate; (c) contacting the detectable conjugate and a solid support; wherein the solid support comprises a recognition moiety; and the recognition moiety binds to the detectable conjugate, thereby forming a bound detectable conjugate; and (d) detecting the bound detectable conjugate, thereby identifying the probe molecule as a ligand for the biological molecule.

In some embodiments, the probe molecules rely on innate chemical reactivity with protein residues. The probe molecule may possess a reactive moiety, such as a photoreactive element, that converts reversible small molecule-protein interactions into stable, covalent adducts upon UV irradiation. The probe molecule may also possess a reporter group, such as an alkyne, which serves as a sterically minimized surrogate reporter allowing late stage conjugation to azide tags by copper-catalyzed azide-alkyne cycloaddition (CuAAK or “click”) chemistry. The probe molecule may also possess a binding element that directs the probe toward proteins that recognize specific structural features.

US published patent applications 2017/0115303 and 2016/0252509 describe examples of probe molecules; each of these publications is hereby incorporated by reference in its entirety, and in particular for the inhibitors of the complement pathway described therein.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given ligand) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The articles “a” and “an” are used herein to refer to one or to more than one (i.e. to at least one) of the grammatical object of the article. By way of example, “an element” means one element or more than one element.

As used herein, the term “interaction”, when referring to an interaction between two molecules, refers to the physical contact (e.g., binding) of the molecules with one another. Generally, such an interaction results in an activity (which produces a biological effect) of one or both of said molecules. The activity may be a direct activity of one or both of the molecules, (e.g., signal transduction).

As used herein, an “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a target polypeptide (e.g., immunoglobulin) or fragment thereof, in which the protein is separated from cellular components of the cells from which it is isolated or recombinantly produced.

In one embodiment, the language “substantially free of cellular material” includes preparations of target protein or fragment thereof, having less than about 30% (by dry weight) of non-target protein (also referred to herein as a “contaminating protein”), more preferably less than about 20% of non-target protein, still more preferably less than about 10% of non-target protein, and most preferably less than about 5% non-target protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “nucleic acid molecule” is intended to include DNA molecules and RNA molecules. A nucleic acid molecule may be single-stranded or double-stranded, but preferably is double-stranded DNA.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

### Screening Assays

The present invention provides a method and system for screening of ligand candidates for biological molecules. The reactive affinity probe interaction discovery (RAPID) is a quantitative binding assay against targets in situ, which sidesteps the challenge of target purification and may provide a systematic approach to discover and target allosteric binding sites.

Disclosed herein, in certain embodiments, is a method of identifying a ligand, comprising: (a) contacting a biological molecule and a probe molecule; wherein the probe molecule comprises a binding element, a reporter group, and a reactive moiety; the probe molecule binds to the biological molecule via the binding element; and the reactive moiety forms a covalent bond with the biological molecule, thereby forming a conjugate; (b) contacting the conjugate and a detectable molecule comprising a functional moiety that reacts with the reporter group, thereby forming a detectable conjugate; (c) contacting the detectable conjugate and a solid support; wherein the solid support comprises a recognition moiety; and the recognition moiety binds to the detectable conjugate, thereby forming a bound detectable conjugate; and (d) detecting the bound detectable conjugate, thereby identifying the probe molecule as a ligand for the biological molecule.

Numerous embodiments are further provided that can be applied to any aspect of the present invention described herein. For example, in some embodiments, the binding element is a small molecule, a peptide, or a nucleic acid (such as RNA or DNA). In some embodiments, the binding element is a component of a library comprising a plurality of binding elements. In some embodiments, the library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, the biological molecule is a protein. In some embodiments, the reactive moiety forms a covalent bond with an amino acid of the protein. In some embodiments, the biological molecule is a lipid, a carbohydrate, or a nucleic acid (such as RNA or DNA). In some embodiments, the biological molecule comprises an epitope tag, for example, FLAG, 6×His, HA, c-myc, glutathione-S-transferase, Strep-tag, maltose-binding protein, chitin-binding protein, S-tag, V5 tag, or AviTag. In some embodiments, the reporter group comprises an azadibenzocyclooctyne, a thiol, an alkene, an alkyne, an azide, a tetrazine, a trans-cyclooctene, a (diphenylphosphino)aryl, (diphenylphosphino)alkyl, or an activated ester (e.g., a hydroxybenzotriazole (HOBt) ester). In some embodiments, the reactive moiety is a photocrosslinker group, a sulfonyl fluoride, a fluorosulfate, a Michael acceptor moiety, a leaving group moiety, or a moiety that forms a covalent bond with a nucleophilic moiety in the side chain of a naturally occurring alpha amino acid (e.g., with the thiol group of a cysteine, the amino group of a lysine, the hydroxyl group of a serine or threonine, or the phenol group of a tyrosine). In some embodiments, the detectable molecule comprises digoxigenin, nickel NTA (nitrilotriacetic acid), a chromophore, or a luminophore. In some embodiments, the chromophore comprises non-fluorochrome chromophore, quencher, an absorption chromophore, fluorophore, organic dye, inorganic dye, metal chelate, or a fluorescent enzyme substrate. In some embodiments, the detectable molecule is biotin. The biotin can be bound to a streptavidin conjugate, such as, a HRP, a SulfoTag, a fluorophore, or a metal chelate. In some embodiments, the functional moiety comprises an azadibenzocyclooctyne, a thiol, an alkene, an alkyne, an azide, a tetrazine, a trans-cyclooctene, a (diphenylphosphino)aryl, (diphenylphosphino)alkyl, or an activated ester (e.g., a hydroxybenzotriazole (HOBt) ester). In some embodiments, the solid support is a membrane, glass, plastic, synthetically prepared polymer, an eppendorf tube, a well of a multi-well plate, or a surface plasmon resonance chip. In some embodiments, the recognition moiety is an antibody, a DNA binding protein, a RNA binding protein, a carbohydrate binding protein, or a lipid binding protein. In some embodiments, the antibody is an antibody against the biological molecule, and/or the epitope tag.

In some embodiments, step (d) comprises detecting the bound detectable conjugates via ELISA, Western blot, immunofluorescence assay, fluorometric assay, fluorometric microvolume assay technology (FMAT), or cell subcellular staining. In some embodiments, the method is performed on a crude cellular extract comprising the biological molecule, performed on a liposomal preparation of proteins comprising the biological molecule, performed on an isolated organelle comprising the biological molecule, performed on a purified protein preparation comprising the biological molecule, or performed in situ. In some embodiments, the method is a cell-based assay. In some embodiments, the biological molecule is expressed in a cell. In some embodiments, the cell is engineered to express the biological molecule. In some embodiments, the cell is lysed prior to step (b). In some embodiments, step (d) further comprises quantifying the amount of the bound detectable conjugate. In some embodiments, step (a) further comprises a substrate for the biological molecule. In some embodiments, the amount of the bound detectable conjugate formed in the presence of the substrate for the biological molecule is less than the amount of the bound detectable conjugate formed in the absence of the substrate (i.e., the probe molecule is a substrate-competitive probe). In other embodiments, the amount of the bound detectable conjugate formed in the presence of the substrate for the biological molecule is greater than the amount of the bound detectable conjugate formed in the absence of the substrate (i.e., the probe molecule is a substrate-cooperative probe).

As used herein, the term “probe” or “test compound” or “candidate agent” refers to an agent or collection of agents (e.g., compounds) that are to be screened for their ability to have an effect on the cell. Test compounds can include a wide variety of different compounds, including chemical compounds, mixtures of chemical compounds, e.g., polysaccharides, small organic or inorganic molecules (e.g., molecules having a molecular weight less than 2000 Daltons, less than 1000 Daltons, less than 1500 Dalton, less than 1000 Daltons, or less than 500 Daltons), biological macromolecules, e.g., peptides, proteins, peptide analogs, and analogs and derivatives thereof, peptidomimetics, nucleic acids, nucleic acid analogs and derivatives, an extract made from biological materials such as bacteria, plants, fungi, or animal cells or tissues, naturally occurring or synthetic compositions.

Depending upon the particular embodiment being practiced, the probes can be provided free in solution, or can be attached to a carrier, or a solid support, e.g., beads. A number of suitable solid supports can be employed for immobilization of the probes. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, probes can be screened individually, or in groups. Group screening is particularly useful where hit rates for effective probes are expected to be low such that one would not expect more than one positive result for a given group.

The properties of small molecules that correlate with good lead compounds are known in the art. Lipinski's Rule of Five provided the original framework for the development of orally bioavailable drug candidates. These rules have been enhanced with the discovery that the number of rotatable bonds (NROT) is an important parameter, a maximum of seven seeming to be optimal for oral bioavailability. The polar surface area (PSA) may be another key property; passively absorbed molecules with a PSA of 110-140 Å2 are thought to have low oral bioavailabilities. Recently, the term ‘lead-like’ was introduced for molecules identified from HTS campaigns that were suitable for optimization and that have properties relatively ‘scaled-down’ in comparison to the Lipinski values. The body of literature is addressing the issues facing compounds that are discovered by screening of drug-size compound libraries. A novel, alternative approach has recently emerged and is referred to as ‘fragment-based’ discovery (Carr, R. and Jhoti, H. (2002) Structure-based screening of low-affinity compounds. Drug Discov. Today 7, 522-527; Erlanson, D. A. et al. (2000) Site-directed ligand discovery. Proc. Natl. Acad. Sci. U.S.A 97, 9367-72; Vetter, D. (2002) Chemical microarrays, fragment diversity, label-free imaging by plasmon resonance-a chemical genomics approach. J. Cell. Biochem. 39, 79-84). Using this approach, the hits identified generally obey a ‘Rule of Three’ and this could be a useful rule for the construction of fragment libraries for lead generation.

This approach begins with fragment libraries (MW 100-250 Da) that are screened using high-throughput X-ray crystallography. These fragments probe key binding interactions in the protein, but are small enough to minimize the chances of unfavourable interactions (electronic or steric) that would prevent them from binding efficiently (Hann, M. et al. (2001) Molecular complexity and its impact on the probability of finding leads for drug discovery. J. Chem. Inf. Comput. Sci. 41, 856-864). The binding modes of these small ligands in the protein are then defined by interpretation of electron density maps. As X-ray crystallography is very effective at identifying weak interactions (μM-mM), fragment hits can be identified that have no measurable activity in a biological assay. Fragment libraries can be constructed to sample chemical diversity or target specific interactions on the protein. Screening of both types of fragment libraries against kinases and proteases, and the subsequent optimization of hits into potent lead compounds indicates that successful hits exhibit particular physicochemical properties.

An analysis of a diverse set of fragment hits indicated that such hits seem to obey, on average, a ‘Rule of Three’, in which molecular weight is <300, the number of hydrogen bond donors is ≤3, the number of hydrogen bond acceptors is ≤3 and C log P is ≤3. In addition, the results suggested NROT (≤3) and PSA (≤60) might also be useful criteria for fragment selection. These data imply that a ‘Rule of Three’ may be useful when constructing fragment libraries for efficient lead discovery.

A number of small molecule libraries are known in the art and commercially available. These small molecule libraries can be screened using the screening methods described herein. A chemical library or compound library is a collection of stored chemicals that can be used in conjunction with the methods described herein to screen candidate agents for a particular effect. A chemical library comprises information regarding the chemical structure, purity, quantity, and physiochemical characteristics of each compound. Compound libraries can be obtained commercially, for example, from Enzo Life Sciences, Aurora Fine Chemicals, Exclusive Chemistry Ltd., ChemDiv, ChemBridge, TimTec Inc., AsisChem, and Princeton Biomolecular Research, among others.

Without limitation, the compounds can be tested at any concentration that can exert an effect on the cells relative to a control over an appropriate time period. In some embodiments, compounds are tested at concentrations in the range of about 0.01 nM to about 100 mM, about 0.1 nM to about 500 microM, about 0.1 microM to about 20 microM, about 0.1 microM to about 10 microM, or about 0.1 microM to about 5 microM.

The compound screening assay can be used in a high through-put screen. High through-put screening is a process in which libraries of compounds are tested for a given activity. High through-put screening seeks to screen large numbers of compounds rapidly and in parallel. For example, using microtiter plates and automated assay equipment, a laboratory can perform as many as 100,000 assays per day, or more, in parallel.

The screening assay can be followed by a subsequent assay to further identify whether the identified test compound has properties desirable for the intended use. For example, the screening assay can be followed by a second assay selected from the group consisting of measurement of any of: bioavailability, toxicity, or pharmacokinetics, but is not limited to these methods.

The present invention also encompasses kits for identifying a ligand as described herein. A kit of the present invention may also include instructional materials disclosing or describing the use of the kit or probes of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

A “kit” is any manufacture (e.g., a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard. One skilled in the art can envision many such controls, including, but not limited to, common molecules. Reagents in the kit may be provided in individual containers or as mixtures of two or more reagents in a single container. In addition, instructional materials which describe the use of the compositions within the kit can be included.

### EXAMPLES

### Example 1: RAPID Protocol for Screening the RAP Library Against an Affinity Tagged Target

### Capture Plate Preparation:

Greiner HiBind plates (Sigma Aldrich Cat. No. M4561-40EA) were coated with capture antibody. The proteinA (Thermo, Cat. No. 101100) was prepared by reconstituting it to 5 mg/mL with 50% glycerol/PBS and diluting it 1:500 into coating buffer (Thermo, BupH™ Carbonate-Bicarbonate Buffer Packs). The plates were washed 1× with 100 uL of coating buffer and placed to the side in sets of six. 50 μL of proteinA was added to each well of every plate. Stamped plates were placed in the 4° C. deli fridge overnight. The next morning, proteinA coated plates were washed 2× with 100 μL of coating buffer. 200 μL of Superblock (Thermo Fisher Cat. No. 37515) was added to each well of every plate and incubate for 1 hour at room temperature. After capture plates have been blocked for 2 hours, all plates were washed 3× with PBS/T. 135 mL of 1 μg/mL capture antibody in SuperBlock buffer was prepared. 50 of the prepared antibody was added to each well of every capture plate and incubated at room temperature for 1 hour.

### Lysate Plate Preparation:

For HEK293T cell line, 7.5M cells per 96-well plate were used. 24 96-well plates were coated with poly-D-lysine (Sigma Aldrich Cat. No. P7280-5MG), and washed. Then, 75K cells were plated per well and incubated overnight to allow the cells to attach. RAP dosing plate were reconstituted with cell imaging media to 4× of the desired final concentration. To each cell plate one at a time, media is removed and 200 μL of cell imaging media (CIM) (Thermo, cat. no. A14291DJ) is dispensed. Next for each plate, the 200 μL of CIM is removed, and immediately 75 μL of CIM is added. Once cells are washed and 75 μL of CIM is dispensed in each plate, 25 μL of the reconstituted RAP is added and the cells are incubated for 30 minutes in a 37° C. cell incubator. While cells incubate, lysis buffer (˜10 mL/plate) is prepared by adding 12.5 mL of 20% DDM (Anatrace Cat. No. D310 25 GM), 250 mL of Hepes buffered saline and 5 complete protease tablets (Sigma Aldrich Cat. No. 4693132001). Plates are irradiated in at a UV crosslinker (Spectrolinker Cat. No. 1195T76). Once irradiated for 3 minutes, media is removed and cells are washed with 200 μL of CIM to remove excess RAP. CIM is then removed out of each plate and 100 μL of lysis buffer is added. Plates are incubated for 1 hr at room temperature to complete lysis.

Reporter biotin is attached to alkyne of RAP using copper catalyzed azide alkyne cycloaddition by making 30 mL of each reagent for a total of 140 mL of click mix. This corresponds to 600 mg THPTA (Click Chemistry Tools Cat. No. 1010-5G), 600 mg Ascorbate (Sigma Aldrich Cat. No. 11140-50G), and 120 mg Copper Sulfate (Sigma Aldrich Cat. No. 451657-10G), and 750 μL of 10 mM picolyl-Biotin-Azide (Click Chemistry Tools Cat. No. 1167-100). Each reagent is individually dissolved in 30 mL of water, then just before starting the click reaction, the reagents are mixed together in the following order: Ting->THPTA->Copper (turns blue)->Ascorbate (turns clear). 40 μL of the click mixture is added to each well of every plate. After 1 hr of incubation, the capture antibody is washed off the capture plates with 3 washes of 300 μL PBS-T (Boston BioProducts Cat. No. IBB-171). The click reactions is quenched by adding 10 μL of 0.5M EDTA (Sigma Aldrich Cat. No. 324506-100ML) per well. 100 μL of lysate is transferred to each corresponding capture plate and incubated for at least 1 hour at room temperature. After the 1 hr capture incubation, the plates are washed 5 times with 300 μL PBS/T.

Streptavidin-HRP (Cell Signaling Technologies Cat. No. 3999S) is prepared in PBS/T by diluting the Cell Signaling Technologies (P/N) material 1:1000. 50 μL of the prepared Streptavidin-HRP is added to each well and incubate for 30 minutes at room temperature. Following the 30 minute streptavidin incubation, the plates are washed 5× with 300 μL PBS/T. The last wash is kept in the plate to prevent drying.

The Tecan is set up with 200 uL tips and the TMB. The trough of the Tecan is filled with at least 135 uL of TMB (Thermo Fisher Cat. No. N301) and the Stamp 50 uL method is opened. Plates are emptied and placed in the Tecan in the appropriate order and the method is run. This process is repeated until all of the plates have received TMB. Each plate is quenched with 50 μL of 0.2N sulfuric acid. The plates are read sequentially on a plate reader quantifying absorbance at 450 nm.

### Identifying Substrate-Sensitive Binders of the Creatine Transporter SLC6A8:

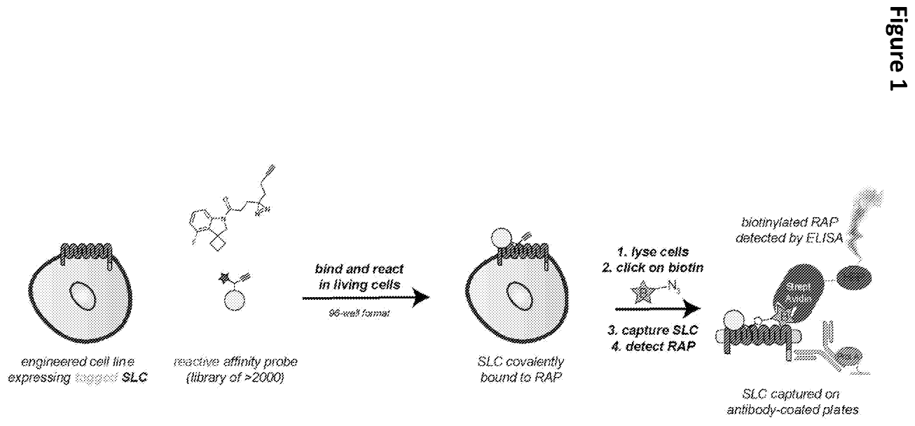

Cells expressing tagged creatine transporter SLC6A8 were used to screen for substrate-sensitive binders. A general protocol for RAPID, a high-throughput screening, in cell binding assay for ligand discovery and target engagement is illustrated in FIG. 1.

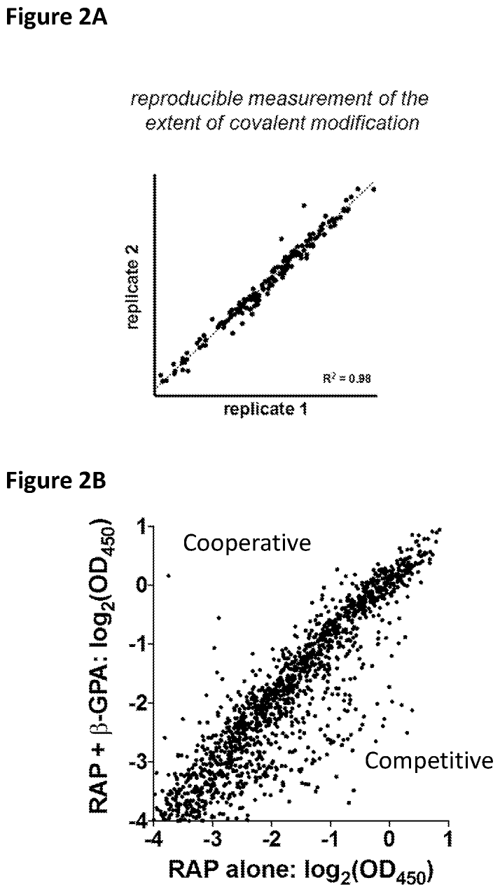

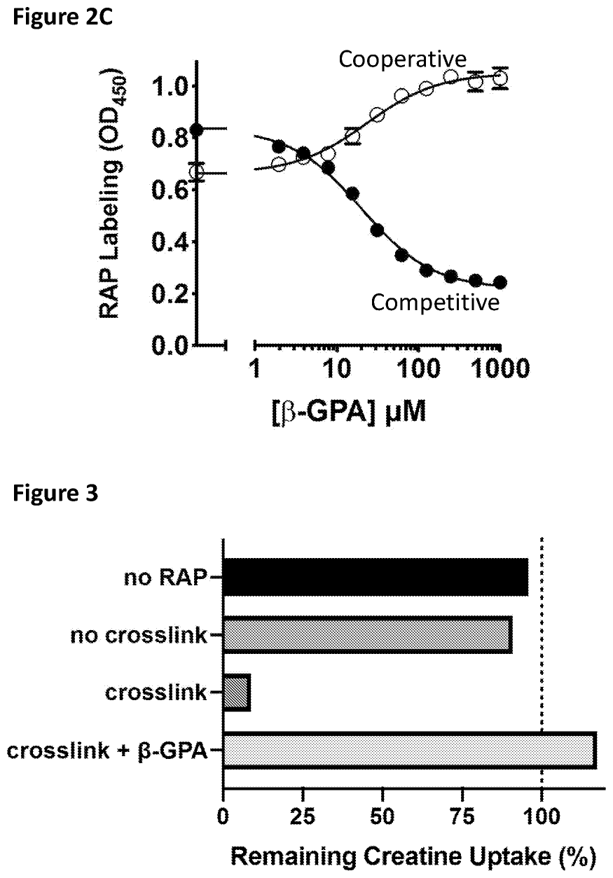

RAPID identifies orthosteric and allosteric ligands as starting points or probes for target occupancy. The extent of covalent modification of the creatine transporter by a subset of the RAP library is highly reproducible (FIG. 2A). Screen of 2000 RAPs against the creatine transporter SLC6A8±the substrate analog guanidinoproprionic acid (GPA) identifies substrate-sensitive binder (FIG. 2B). Dots that fall off-diagonal are either substrate-competitive or substrate-cooperative. Two RAPs identified from the screen show dose-dependent inhibition or enhancement of covalent modification of the target as a function of the concentration of GPA (FIG. 2C). The IC50/EC50 corresponds to GPA's known inhibition constant (˜30 Cells were dosed for 30 minutes with 100 μM JN-1724 with or without 1 mM β-GPA and then irradiated for 6 minutes with 365 nm light. Cells were washed and the residual transport activity of SLC6A8 was measured via a creatine uptake assay. covalent inactivation of the creatine transporter SLC6A8 by the reactive affinity probe JN-1724 and protection by co-dosing with competitor β-GPA is shown in FIG. 3.

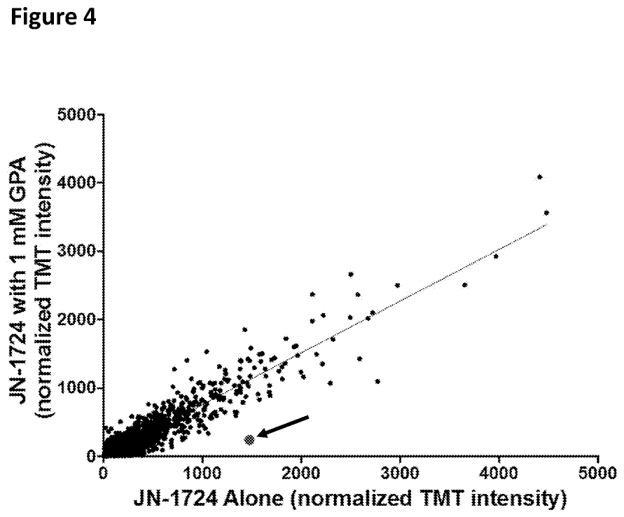

Treatment of cells with substrate-competitive RAP quantitatively inhibits the creatine transporter. A 30 minute 100 μM dose of JN-1724 followed by 6 minutes of cross-linking at 365 nm is enough to inhibit SLC6A8 transport of creatine to 5.7% of normal transport. To confirm target engagement by unbiased mass spectrometry of the adductome, Cells were dosed for 30 minutes with 20 μM JN-1724 with or without 1 mM β-GPA and then irradiated for 6 minutes with 365 nm light. Cells were lysed; biotin was clicked on; and biotinylated proteins were affinity-purified with streptavidin, digested with trypsin, and identified by tandem mass spectrometry with TMT quantification. SLC6A8 (dot pointed at with arrow) was one of the proteins identified and co-dosing with 1 mM β-GPA reduced the level of enrichment by 80% (FIG. 4).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of identifying a ligand, comprising:
(a) contacting a biological molecule and a probe molecule; wherein the probe molecule comprises a binding element, a reporter group, and a reactive moiety; the probe molecule binds to the biological molecule via the binding element; and the reactive moiety forms a covalent bond with the biological molecule, thereby forming a conjugate;
(b) contacting the conjugate and a detectable molecule comprising a functional moiety that reacts with the reporter group, thereby forming a detectable conjugate;
(c) contacting the detectable conjugate and a solid support; wherein the solid support comprises a recognition moiety; and the recognition moiety binds to the detectable conjugate, thereby forming a bound detectable conjugate; and
(d) detecting the bound detectable conjugate, thereby identifying the probe molecule as a ligand for the biological molecule.

2. The method of claim 1, wherein the binding element is a small molecule.

3. The method of claim 1, wherein the binding element is a peptide.

4. The method of claim 1, wherein the binding element is a nucleic acid.

5. The method of claim 4, wherein the nucleic acid is RNA or DNA.

6. The method of any one of claims 1-5, wherein the binding element is a component of a library comprising a plurality of binding elements.

7. The method of claim 6, wherein the binding elements are small-molecule fragments; and each small-molecule fragment has at least one of the following characteristics: molecular weight ≤300 Da, c Log P ≤3, hydrogen bond donors ≤3, and hydrogen bond acceptors ≤3.

8. The method of any one of claims 1-7, wherein the biological molecule is a protein.

9. The method of claim 8, wherein the reactive moiety forms a covalent bond with an amino acid of the protein.

10. The method of any one of claims 1-7, wherein the biological molecule is a lipid.

11. The method of any one of claims 1-7, wherein the biological molecule is a carbohydrate.

12. The method of any one of claims 1-7, wherein the biological molecule is a nucleic acid.

13. The method of claim 12, wherein the nucleic acid is RNA.

14. The method of claim 12, wherein the nucleic acid is DNA.

15. The method of any one of claims 1-14, wherein the biological molecule comprises an epitope tag.

16. The method of claim 15, wherein the epitope tag is FLAG, 6×His, HA, c-myc, glutathione-S-transferase, Strep-tag, maltose-binding protein, chitin-binding protein, S-tag, V5 tag, or AviTag.

17. The method of any one of claims 1-16, wherein the reporter group comprises an azadibenzocyclooctyne, a thiol, an alkene, an alkyne, an azide, a tetrazine, a trans-cyclooctene, a (diphenylphosphino)aryl, (diphenylphosphino)alkyl, or an activated ester (e.g., a hydroxybenzotriazole (HOBt) ester).

18. The method of any one of claims 1-17, wherein the reactive moiety is a photocrosslinker group, a sulfonyl fluoride, a fluorosulfate, a Michael acceptor moiety, a leaving group moiety, or a moiety that forms a covalent bond with a nucleophilic moiety in the side chain of a naturally occurring alpha amino acid (e.g., with the thiol group of a cysteine, the amino group of a lysine, the hydroxyl group of a serine or threonine, or the phenol group of a tyrosine).

19. The method of any one of claims 1-18, wherein the detectable molecule comprises digoxigenin, nickel NTA (nitrilotriacetic acid), a chromophore, or a luminophore.

20. The method of claim 19, wherein the detectable molecule comprises a digoxigenin.

21. The method of claim 19, wherein the detectable molecule comprises a nickel NTA (nitrilotriacetic acid).

22. The method of claim 19, wherein the detectable molecule comprises a chromophore.

23. The method of claim 22, wherein the chromophore comprises non-fluorochrome chromophore, quencher, an absorption chromophore, fluorophore, organic dye, inorganic dye, metal chelate, or a fluorescent enzyme substrate.

24. The method of claim 19, wherein the detectable molecule comprises a luminophore.

25. The method of any one of claims 1-18, wherein the detectable molecule is biotin.

26. The method of claim 25, wherein the biotin is bound to a streptavidin conjugate.

27. The method of claim 26, wherein the streptavidin conjugate comprises a HRP, a SulfoTag, a fluorophore, or a metal chelate.

28. The method of any one of claims 1-27, wherein the functional moiety comprises an azadibenzocyclooctyne, a thiol, an alkene, an alkyne, an azide, a tetrazine, a trans-cyclooctene, a (diphenylphosphino)aryl, (diphenylphosphino)alkyl, or an activated ester (e.g., a hydroxybenzotriazole (HOBt) ester).

29. The method of any one of claims 1-28, wherein the solid support is a membrane, glass, plastic, synthetically prepared polymer, an eppendorf tube, a well of a multi-well plate, or a surface plasmon resonance chip.

30. The method of any one of claims 1-29, wherein the recognition moiety is an antibody, a DNA binding protein, a RNA binding protein, a carbohydrate binding protein, or a lipid binding protein.

31. The method of claim 30, wherein the recognition moiety is an antibody.

32. The method of claim 31, wherein the antibody is an antibody against the biological molecule.

33. The method of claim 31, wherein the antibody is an antibody against the epitope tag.

34. The method of any one of claims 1-33, wherein step (d) comprises detecting the bound detectable conjugates via ELISA.

35. The method of any one of claims 1-33, wherein step (d) comprises detecting the bound detectable conjugates via Western blot.

36. The method of any one of claims 1-33, wherein step (d) comprises detecting the bound detectable conjugates via immunofluorescence assay.

37. The method of any one of claims 1-33, wherein step (d) comprises detecting the bound detectable conjugates via fluorometric assay.

38. The method of any one of claims 1-33, wherein step (d) comprises detecting the bound detectable conjugates via fluorometric microvolume assay technology (FMAT).

39. The method of any one of claims 1-33, wherein step (d) comprises detecting the bound detectable conjugates via cell subcellular staining.

40. The method of any one of claims 1-39, wherein the method is performed on a crude cellular extract comprising the biological molecule.

41. The method of any one of claims 1-39, wherein the method is performed on a liposomal preparation of proteins comprising the biological molecule.

42. The method of any one of claims 1-39, wherein the method is performed on an isolated organelle comprising the biological molecule.

43. The method of any one of claims 1-39, wherein the method is performed on a purified protein preparation comprising the biological molecule.

44. The method of any one of claims 1-39, wherein the method is performed in situ.

45. The method of any one of claims 1-39, wherein the method is a cell-based assay.

46. The method of claim 45, wherein the biological molecule is expressed in a cell.

47. The method of claim 46, wherein the cell is engineered to express the biological molecule.

48. The method of any one of claims 45-47, wherein the cell is lysed prior to step (b).

49. The method of any one of claims 1-48, wherein step (d) further comprises quantifying the amount of the bound detectable conjugate.

50. The method of claim 49, wherein step (a) further comprises a substrate for the biological molecule.

51. The method of claim 50, wherein the amount of the bound detectable conjugate formed in the presence of the substrate for the biological molecule is less than the amount of the bound detectable conjugate formed in the absence of the substrate (i.e., the probe molecule is a substrate-competitive probe).

52. The method of claim 50, wherein the amount of the bound detectable conjugate formed in the presence of the substrate for the biological molecule is greater than the amount of the bound detectable conjugate formed in the absence of the substrate (i.e., the probe molecule is a substrate-cooperative probe).
